(19) United States
(12) Patent Application Publication
(10) Pub. No.: US 2012/0265081 A1
(43) Pub. Date: Oct. 18, 2012

(54) Diagnostic Method

(76) Inventor: Ben Jun Wu, Markham (CA)

(21) Appl. No.: 13/085,757

(22) Filed: Apr. 13, 2011

(51) Int. Cl. A61B 5/022; A61B 5/00; A61B 5/021

(52) U.S. Cl. 600/490; 600/485; 600/300

(57) ABSTRACT

This is a diagnostic method to determine and predict disease and observe the development of a disease by using a dual-arm blood pressure monitor that measures the balance of relationship between human organs and functions.

This method uses the dialectical theory of “the balance of yin and yang’ in traditional Chinese medicine and is combined with modern blood pressure measuring tools, and is able to forecast and observe the process and effects of treatment of a disease. The method makes it possible for efficacy of Chinese medicine diagnosis go out of fuzzy situation, and is a breakthrough in disease forecast, diagnosis, and treatment which is also considered a significant contribution to Western medicine for diagnostics.

### TECHNICAL AREA

This invention is one kind of new diagnosis method for disease, medically using for measuring blood pressure and blood stream of human body by blood pressure meter as medical diagnosis instrument.

### TECHNICAL BACKGROUND

The dialectical theory of “the balance of yin and yang” is the basis for traditional Chinese medicine, and is the basic knowledge in Chinese medicine to perceive human's health status. Chinese medicine believes: the “yin and yang” in human body are keeping in a state of balance, breaking the balance of yin and yang will lead to either positivity or negativity, resulting in a pathological state. In the cause, the Chinese medicine practitioners believe that the occurrence of the disease, fundamentally speaking, is due to that the balance of yin and yang is destroyed, and there have been positivity partial failure. Traditional Chinese medicine's “balance of yin and yang” theory of dialectics has guided the Chinese medicine for thousands of years.

A technical problem in the use of Chinese medicine's “the balance of yin and yang” theory of disease diagnosis lies in how to pinpoint the human body and the balance and imbalance between yin and yang. The blood pressure data has already been recognized as an important indicator of human health by both Chinese medicine and Western medicine. However, traditional Chinese medicine diagnoses the balance of yin and yang by “feeling the patient's pulse”. This method requires the extensive experiences of a doctor, is hard to be grasped, and lack of accuracy.

The invention of blood-pressure meter in Western medicine provides an efficient tool for the diagnosis of cardiovascular diseases. However, in a long period, blood-pressure meter has always been basically used for diagnosis of cardiovascular diseases, such as high or low blood pressure, but data in blood pressure measurement has not been fully recognized, and hasn't been used in conjunction with the Chinese yin-yang balance theory in the determination of balance and imbalance of human body. For the past time, the blood-pressure meter has been the device only used for the measurement of blood pressure through single arm of human body, due to the theory that the data of human blood pressure measured, being gained from blood-pressure meter, may reflect the human body and balance or imbalance of human body has not been fully recognized. Actually, the data from two arms of the same human body are some different, and thus many information may be gained through the blood pressure data.

### Content of Invention

The inventor creatively utilizes the method of blood pressure measurement through dual-arm of human body, and through long-term clinical practice, found that variation in statistics on human body may reflect the balance and imbalance between human organs, i.e. the Chinese medicine theory of yin and yang balance and imbalance, and reflect that it may or may have been suffering from certain diseases. In the course of the treatment of the disease, the reflection of treatment process to human body may be observed through continuous measurement of blood pressures on dual-arm of human body. After one treatment phase, the effect of treatment may be learned through the comparison of data measured prior- and post-treatment through blood pressures on dual-arm of human body.

Utilizing the method of blood pressure measurement through dual-arm of human body for diagnosis of disease is one of the important technical characteristics of this invention. Single arm measurement has always been utilized mainly for diagnosis of whether suffering from cardiovascular diseases. The invention is to measure blood pressure using blood pressure meter on dual-arm of human body.

Inventor discovered in clinical practice that the blood pressure data of dual-arm and single arm of human body are not completely the same at the same time, these different data may reflect a lot of states of human body. The different data is very important, it is the major evidence for diagnosing balance or imbalance between various parts of human body, and is also one of the technical characteristics of this invention.

### EXAMPLE 1

Patient 7005: measured at May 28, 2008. data are shown below:

| | | | | |
|---|---|---|---|---|
| First measure: | left arm: | 166/96/97 | right arm: | 173/99/105 |
| Second measure: | left arm: | 159/101/100 | right arm: | 158/95/100 |

This clearly shows that the patient has different statistics in arm blood pressure, which means that imbalance exists between body organs and functions, and the patient may be suffering from a disease who is taking seven Western medicines. The inventor uses some Chinese medical treatment methods (kept confidential) on the patient.

Measured at Jun. 22, 2008, data shows:

| | | | | |
|---|---|---|---|---|
| First measure: | left arm: | 139/85/85 | right arm: | 158/92/92 |
| Second measure: | left arm: | 141/85/86 | right arm: | 144/90/92 |

Dual-arm pressure measures show that the treatment has shown positive results, and the patient also feels better. And further treatment is used:

Measured at Jul. 6, 2008, data shows:

| | | | | |
|---|---|---|---|---|
| First measure: | left arm: | 139/80/90 | right arm: | 144/87/90 |
| Second measure: | left arm: | 137/82/90 | right arm: | 144/72/90 |

Data shows that the patient is in the process of recovery, and western medicine has been reduced to half amount.

Measured at Sep. 7, 2008, data shows:

| | | | | |
|---|---|---|---|---|
| First measure: | left arm: | 140/85/77 | right arm: | 127/79/77 |
| Second measure: | left arm: | 136/84/77 | right arm: | 128/78/80 |

Data shows that the patient is further recovering, and western medicine has been ceased. The patient feels in a perfect condition.

### EXAMPLE 2

Patient 6596: measured at Feb. 17, 2008, data are shown below:

| | | | | |
|---|---|---|---|---|
| First measure: | left arm: | 183/121/74 | right arm: | 178/98/77 |
| Second measure: | left arm: | 205/183/70 | right arm: | 186/103/74 |

Data shows that severe imbalance exists in the patient, represents suffering from multiple diseases, which may also cause other diseases.

Measured at Apr. 27, 2008 (treatment for six times, medical approaches are kept confidential), data shows:

| | | | | |
|---|---|---|---|---|
| First measure: | left arm: | 146/83/73 | right arm: | 176/140/79 |
| Second measure: | left arm: | 166/95/77 | right arm: | 183/143/86 |

Data shows that the patient is in the process of recovery.

Measured at May 17, 2008 (treatment for twice), data shows:

| | | | | |
|---|---|---|---|---|
| First measure: | left arm: | 133/94/77 | right arm: | 177/98/75 |
| Second measure: | left arm: | 162/93/75 | right arm: | 179/97/74 |

Data shows that the patient is further recovering, and the western medicine has been continuously ceased.

Measured at Jul. 21, 2008, the specific data of blood pressure shows:

| | | | | |
|---|---|---|---|---|
| First measure: | left arm: | 140/88/86 | right arm: | 130/91/85 |
| Second measure: | left arm: | 140/81/85 | right arm: | 165/94/86 |

The data shows that the state of patient has been significantly improved, body weight has been increased.

Measured at Nov. 4, 2008 (treatment for four times), data shows:

| | | | | |
|---|---|---|---|---|
| First measure: | left arm: | 148/84/74 | right arm: | 153/91/76 |
| Second measure: | left arm: | 153/87/75 | right arm: | 142/94/76 |

Through the dual-arm blood pressure measurement, the state of patient has been significantly improved, the original constipation has become normal, and the initial low back pain, lower limb pain, and sleep problems have been significantly improved, physical health status has become restored, without taking any western medicine for this whole period.

To protect the secret of the invention, the inventor of the invention doesn't disclose (1) the specific meanings of the dual-arm blood pressure measurement data of the above-mentioned two cases of patients; (2) the specific disease that the diagnosis has been performed through the dual-arm blood pressure measurement data; (3) the specific type of drugs that should be taken according to the dual-arm blood pressure measurement data.

The inventor of the dual-arm blood pressure measurement reflects the balance or imbalance between human organs and functions through the data measured by blood pressured of dual-arm, i.e. the balance or imbalance of yin and yang of human body in Chinese medicine. The significance of the invention is: to change the method of diagnosis relying on the long-term experience, and determine blood pressure and blood stream state of a patient through feeling the pulse of traditional Chinese medicine, into the method of measuring dual-arm through blood pressure meter to accurately diagnose the balance or imbalance between human organs and functions. The invention enhances the accuracy and scientific of Chinese medicine diagnosis, observation and testing efficacy, with great significance.

Traditional Chinese medicine adopted more or less vein blood pressure and blood flow of patients to determine the situation and thus the disease that a patient is suffering from may be found. Chinese medicine's perform diagnosis through the method of feeling the pulse is correct, but this kind of a method (1) requires rich experiences and long term clinical practice of a Chinese medicine practitioner (2) is not precise enough. Therefore, several different diagnosis may be gained when feeling the pulse is performed by several practitioners. Through this invention, just to know how to use the existing blood pressure is enough, and no long-term clinical experience is required. Additionally, the accuracy of a blood pressure meter may significantly decrease the error rate of diagnosis.

Chinese medicine theory insists that the inter-coordination of “yin and yang” may maintain the relative balance of organs and functions of human body, and is the basis of physiological function of human body. Immunology of modern medicine has also gradually recognized that the immune system of human body is consisted of immune organs, immune cells and immune molecules. Thymus, bone marrow, lymph nodes, spleen and peripheral lymphoid tissue and immune ecosystems (immune cells of various subsystems, cell subsystems, subsystems antibodies, cytokines subsystems, humoral factors subsystem, environment subsystem) together constitute the body immune defense system. Their immune response interdependence and mutual restraint. This theory of Western medicine and Chinese medicine theory of yin and yang balance has been very close. Through determining blood pressure and blood stream of a patient by feeling the pulse, traditional Chinese medicine determines the status of the human body and so the body to determine whether the function of organs and balance (the balance of yin and yang), and then determine the patient has suffered from in addition to suffering from diseases may be suffering from other diseases in future. However, it might be difficult for traditional Chinese medicine to forecast the disease of human body only through feeling the pulse. In fact, Western medicine, although with many modern Western equipments, is also difficult to predict the disease of human body, on account of the lack of the Chinese medicine theory of observing the human body as a whole. The difficulty lies in whether a more precise way to know where the body's organs and functions of the imbalance, which is part of the body which the imbalance of yin and yang, or which part of the body organ function and loss of balance caused by a disorder of the immune system. Basing on the method of measuring blood pressure on dual-arm of human body, this invention may be more accurately determine that the human body organs and functions of balance and imbalance, that is, the balance of “yin and yang” theory of Chinese medicine or Western medicine or disorder and said a complete system, so as to correctly predict the probability of disease.

Long term clinical experience may also be required and inaccuracy issue may also be existed in determination when traditional Chinese medicine is utilized through determining blood pressure and blood stream of a patient by feeling the pulse to observe the process of treatment. This invention may significantly improve the treatment effect only through continuously observing the change in blood pressure data measured through dual-arm of a patient to learn the treatment state, which may increase the treatment effect rapidly. Some people hold prejudice on traditional Chinese medicine treatment process, one of the reasons is that Chinese medicine may not demonstrate the effect through displaying data in time, and the confirmation of specific method that takes effect at last is also an issue when Chinese and western medicine, or several methods have been utilized at the same time. Since this invention may use data measured by blood pressure meter continuously to demonstrate the effect of the treatment process, the treatment is more effective and accurate. Therefore, it may be easier to identify the more effective method when multiple methods have been used.

1, This is a diagnostic method to determine and predict disease and observe the development of a disease by using a dual-arm sphygmomanometer that measures the balance of relationship between human organs and functions. The method includes the following processes: (1) to measure dual-arm blood pressure by using a sphygmomanometer; (2) to analyze the data obtained from the dual-arm blood pressure measurements; (3) to determine whether or not the relationship between human organs and functions is in a balanced state based on the analysis in dual-arm blood pressure measurements; (4) to diagnose any potential diseases that the patient is suffering from by looking at whether the relationship between human organs and functions is in a balanced state; (5) to forecast any potential disease that the patient my likely suffer from; (6) to measure dual-arm blood pressures on a regular basis throughout the course of treatment with an attempt to observe the treatment process; (7) to obtain treatment results based on the dual-arm blood pressure variance measured during the course of treatment.

2, The balance of relationship of human organs and functions refers to the balance of “yin and yang” in traditional Chinese medicine.

3, According to the method specified in Article 1 of the claims, it is found that measurements on different arms at the same time may vary. These data are very important, as they are able to reflect the balance and imbalance of relationship of human organs and functions and are critical for diagnosis of the state of human.

4, According to the method specified in Article 1 of the claims, this invention uses sphygmomanometer to measure blood pressure in both arms instead of a single arm. The following descriptions related to human arm blood pressure.

5, measurements all refer to the common measurement of blood pressure. According to the method specified in Article 1 of the claims, this invention is used to analyze the blood pressure measurements of both arms instead of single arm;

6, According to the method specified in Article 1 of the claims, this invention uses arm blood pressure measurements to analyze the balance and imbalance of relationship of human organs and functions, such balance also refers to the balance of yin and yang as specified in Traditional Chinese Medicine;

7, According to the method specified in Article 1 of the claims, the invention uses arm blood pressure measurements to analyze the blood circulation in human organs;

8, According to the method specified in Article 1 of the claims, the invention uses arm blood pressure measurements to analyze the condition of human organs;

9, According to the method specified in Article 1 of the claims, the invention uses arm blood pressure measurements to analyze the blood circulation and condition of human organs to predict the future of human organs that may be suffering from certain diseases or have been hidden suffering from certain diseases factors,

10, According to the method of diagnosis described in this claim, the invention of the human arm blood pressure measurements, including body parts, organs analysis of the relationship between the balance is smooth and other relations, the diagnosis of human organs may be suffering from certain diseases

11, According to the method of diagnosis described in this claim, the invention of the human arm blood pressure measurements, in the course of the treatment of the disease can be treated by comparing measurements of blood pressure in the process of observing and monitoring the changes in the course of treatment;

12, According to the method of diagnosis described in this claim, the invention of the human arm blood pressure measurements in the treatment of the disease in the whole process can be compared before treatment, during treatment and completed treatment for a blood pressure measurements to understand the changes in treatment.
